(19) United States
(12) Patent Application Publication
(10) Pub. No.: US 2024/0293656 A1
(43) Pub. Date: Sep. 5, 2024

(54) FUNCTIONAL ADAPTER FOR DIALYSIS MACHINE

(71) Applicant: Harvey Kertland, Cedar Park, TX (US)

(72) Inventor: Harvey Kertland, Cedar Park, TX (US)

(21) Appl. No.: 18/116,671

(22) Filed: Mar. 2, 2023

(51) Int. Cl. A61M 39/10

(52) U.S. Cl. CPC A61M 39/10

(57) ABSTRACT

Dialysis machines today, are of a substandard design as the faucet point is threaded and not entirely flush with the tubing and water pressure from the tap which could potentially force the entire apparatus to fall off in the middle of a dialysis treatment.

This invention ultimately serve as a supplementary device, does not claim any design rights from any dialysis machine used, this device is intended to thread to the faucet, utilizing the components: faucet adapter, garden hose swivel, garden hose adapter and metal quick connect. All these components ensures a precise connection will protect the adapter from being disconnected during normal operation of a dialysis machine at all varying psi.

### CROSS-REFERENCE TO RELATED APPLICATIONS

This application claims the benefit of no United States Provisional Applications filed.

### BACKGROUND OF INVENTION

This invention relates in general to the creation of specific purpose plumbing device used as a connection to the water source of a dialysis machine.

### BRIEF DESCRIPTION OF THE DRAWINGS

FIG. 1 is a diagrammatic representation of the functional adapter for the dialysis machine.

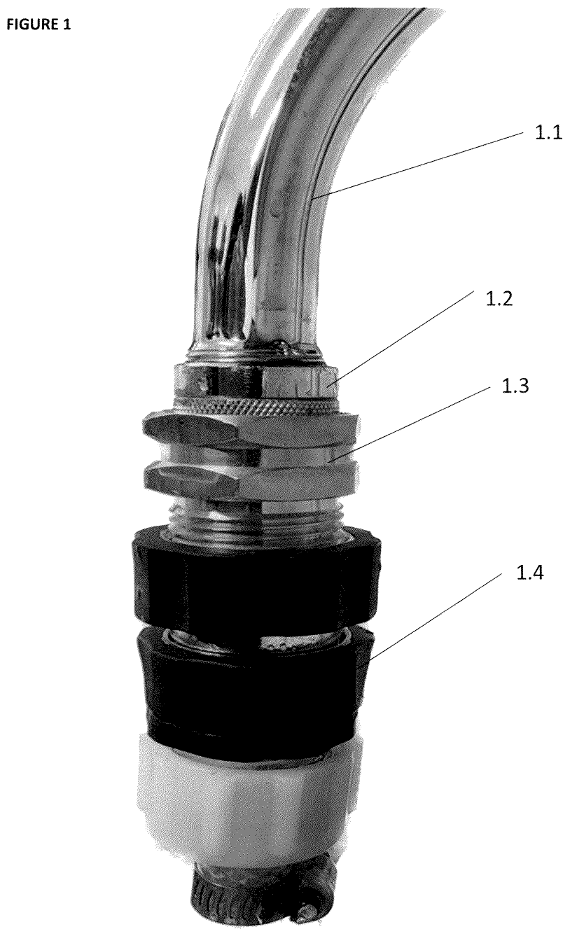

What is claimed is:

1. The method for connecting to the dialysis machine with all the components of the invention, which includes:
1.1. Water source connector for the dialysis machine;
1.2. Faucet Adapter (female 55/64″−27 to male ¾″ hose);
1.3. Garden Hose Adapter (¾″GHT Female×¾″ NPT Male Connector);
1.4. Garden Hose Quick Release Adapter;
FIG. 2 is a diagrammatic representation of the functional adapter for the dialysis machine.

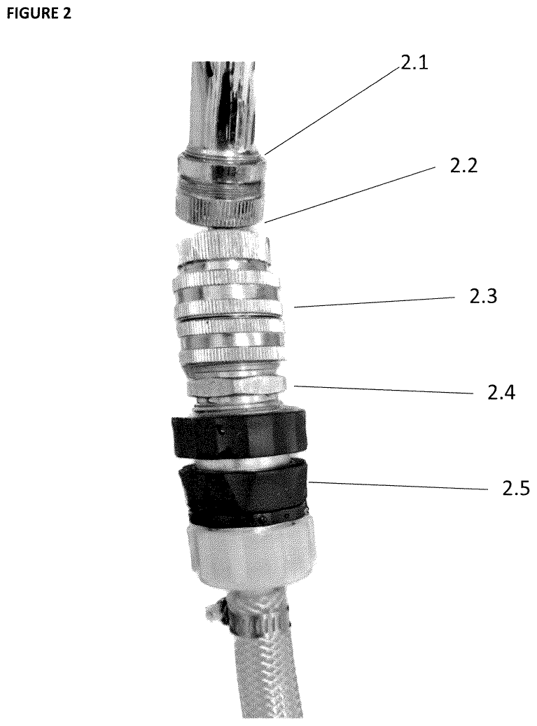

2. The method for connecting to the dialysis machine with all the components of the invention, including an additional variant to the design which encompass a swivel two-part function. The parts list includes:
2.1. Water source connector for the dialysis machine;
2.2. Swivel Faucet Adapter (female 55/64″−27 to male ¾″ hose);
2.3. Garden Hose Adapter (¾″ GHT Female×¾″ GHT Female Connector);
2.4. Garden Hose Adapter (¾″ GHT Male×¾″ NPT Male Connector);
2.5. Garden Hose Quick Release Adapter;
FIG. 3 is a diagrammatic representation of the functional adapter for the dialysis machine.

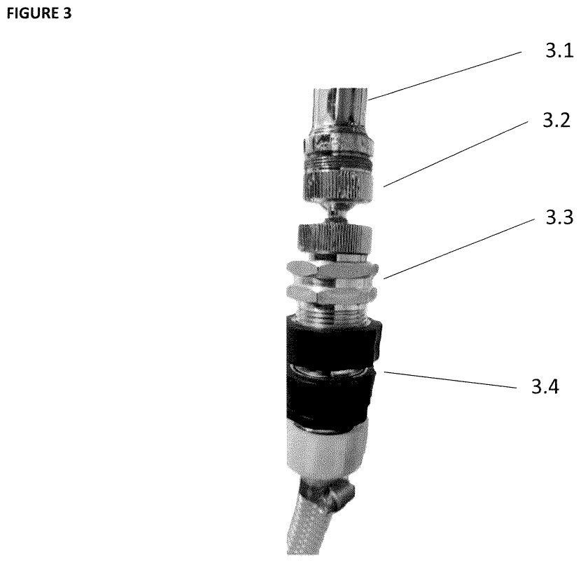

3. The method for connecting to the dialysis machine with all the components of the invention, including an additional variant to the design which encompass a swivel one-part function. The parts list includes:
3.1. Water source connector for the dialysis machine;
3.2. Swivel Faucet Adapter (female 55/64″−27 to male ¾″ hose);
3.3. Garden Hose Adapter (¾″ GHT Female×¾″ NPT Male Connector);
3.4. Garden Hose Quick Release Adapter;
FIG. 4 is a diagrammatic representation of the functional adapter for the dialysis machine.

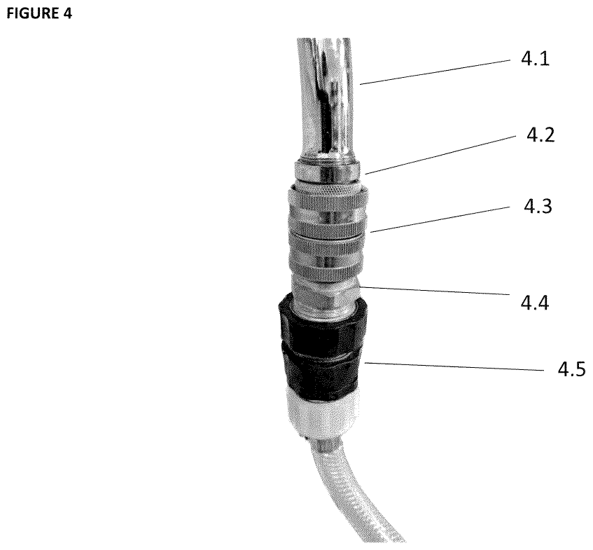

4. The method for connecting to the dialysis machine with all the components of the invention, including an additional variant to the design which encompass a non-swivel two-part connector, function. The parts list includes:
4.1. Water source connector for the dialysis machine;
4.2. Non-Swivel Faucet Adapter (female 55/64″−27 to male ¾″ hose);
4.3. Garden Hose Adapter (¾″ GHT Female×¾″ GHT Female Connector);
4.4. Garden Hose Adapter (¾″ GHT Male×¾″ NPT Male Connector);
4.5. Garden Hose Quick Release Adapter;
